(19) United States
(12) Patent Application Publication
(10) Pub. No.: US 2024/0196824 A1
(43) Pub. Date: Jun. 20, 2024

(54) BRASSICA INTERGENERIC CROSSBRED PLANT CONTAINING LARGE AMOUNT OF GLUCORAPHANIN, AND CREATION METHOD THEREFOR

(71) Applicants: NATIONAL AGRICULTURE AND FOOD RESEARCH ORGANIZATION, Tsukuba-shi, Ibaraki (JP); KAGOME CO., LTD., Nagoya-shi, Aichi (JP)

(72) Inventors: Tomohiro KAKIZAKI, Tsu-shi, Mie (JP); Takayoshi OHARA, Tsu-shi, Mie (JP); Etsuko ITABASHI, Tsu-shi, Mie (JP); Mitsuyo KAWASAKI, Tsu-shi, Mie (JP); Ryota ENDO, Nasushiobara-shi, Tochigi (JP); Yusuke USHIDA, Nasushiobara-shi, Tochigi (JP)

(73) Assignees: NATIONAL AGRICULTURE AND FOOD RESEARCH ORGANIZATION, Tsukuba-shi, Ibaraki (JP); KAGOME CO., LTD., Nagoya-shi, Aichi (JP)

(21) Appl. No.: 18/552,246

(22) PCT Filed: Mar. 22, 2022
(86) PCT No.: PCT/JP2022/013175

§ 371 Date: Sep. 25, 2023

(30) Foreign Application Priority Data
Apr. 1, 2021 (JP) 2021-063196

(51) Int. Cl. A01H 1/02; A01H 1/00; A01H 6/20

(52) U.S. Cl. CPC A01H 1/021; A01H 1/10; A01H 6/203; A01H 6/206

(57) ABSTRACT

A Brassicaceae plant containing a large amount of glucoraphanin is obtained. A first parent plant and a second parent plant, which are both Brassicaceae plants classified into different genera, are crossbred, thereby obtaining an intergeneric crossbred plant. The first parent plant has 5 mg/100 g (fresh weight) or more of glucoraphanin. The second parent plant has a loss-of-function type glucoraphasatin synthase gene.

### TECHNICAL FIELD

The present invention relates to a Brassicaceae intergeneric crossbred plant comprising a large amount of glucoraphanin and a creation method therefor.

### BACKGROUND ART

Sulforaphane, which is a kind of isothiocyanate, is a phytochemical comprised in plants of the family Brassicaceae, particularly in Brassica oleracea. Sulforaphane is a functional component known to have bioactivities such as anti-cancer effects by activating the production of detoxification enzymes in the human body, as well as liver function-enhancing and antioxidant effects (see, for example, Non Patent Literature 1).

In plant cells, sulforaphane normally exists in the form of glucoraphanin as a precursor. Glucoraphanin is a type of secondary metabolite glucosinolates, also called mustard oil glycosides. Glucoraphanin in the cells is exposed outside the cells by mastication or the like, reacts with myrosinase, an enzyme endogenous in the plant body, or is degraded by intestinal bacteria to convert to sulforaphane.

Patent Literature 1 discloses a method for obtaining a Brassica cultivar comprising a large amount of glucoraphanin, the method comprising crossbreeding a wild-type Brassica species with a Brassica oleracea breeding line and selecting a crossbreed having a higher amount of 4-methylsulfinylbutyl glucosinolate (glucoraphanin) than the original breeding line. Patent Literature 2 discloses a Brassica oleracea plant comprising a large amount of glucosinolate, the plant comprising a Myb28 allele from Brassica villosa and lacking an ELONG allele from Brassica villosa genetically linked to the Myb allele.

In general, the genus Raphanus of the family Brassicaceae does not comprise glucoraphanin at an available level. Patent Literature 3 discloses a method for producing a Japanese radish line comprising a large amount of glucoraphanin by selecting an individual comprising a large amount of glucoerucin and 4-methylthio-3-butenyl glucosinolate (glucoraphasatin) in an amount ⅕ or less the amount of glucoerucin and carrying out self-reproduction.

Non Patent Literature 2 reports that glucoraphanin and glucoraphenin are detected at high contents in Raphanobrasica, which is an intergeneric crossbreed of Japanese radish of the genus Raphanus of the family Brassicaceae and kale of the genus Brassica.

It is known that glucoraphasatin usually accounts for 90% or more of the glucosinolates comprised in Japanese radish. Non Patent Literature 3 reports that the glucoraphasatin synthase (GRS) gene was identified. In addition, Patent Literature 4 discloses a method for obtaining a Japanese radish line comprising a small amount of glucoraphasatin by crossbreeding Japanese radish individuals having the loss-of-function type GRS gene to reduce the peculiar odor and yellowing of Japanese radish from degradation products of glucoraphasatin.

### PRIOR ART

### Patent Literature

Patent Literature 1: JP 2002-511235 A

Patent Literature 2: JP 2014-76045 A

Patent Literature 3: JP 2012-110238 A

Patent Literature 4: JP 2016-86761 A

### Non Patent Literature

Non Patent Literature 1: Zhang, Y. et al., P. Proc. Natl. Acad. Sci., Vol.91, pp. 3147-3150 (1994)

Non Patent Literature 2: Research information on the website of the Nagano Agricultural Experiment Station, Research result, “Technical information,” [Result title] Raphanobrasica “Naga-no No. 48” is promising as a new vegetable comprising many functional ingredients (URL https://www.agries-nagano.jp/wp/wp-content/uploads/2019/04/2018-2-g10.pdf)

Non Patent Literature 3: Kakizaki, T. et. al., Plant Physiology, Vol. 173, pp. 1583-1593 (2017)

### SUMMARY OF INVENTION

### Object to be Achieved by the Invention

An object of the present invention is to obtain a Brassicaceae plant comprising a large amount of glucoraphanin.

### Means for Achieving the Object

As a result of conducting diligent studies to achieve the object described above, the present inventors have found that a glucoraphanin content is increased in an intergeneric crossbreed obtained by crossbreeding a Brassica plant and a Raphanus plant having the loss-of-function type GRS gene. This has led to the completion of the present invention.

The present invention encompasses the following.
(1) An intergeneric crossbred plant from a Brassica plant and a Raphanus plant, for which a ratio of glucoraphanin content to glucoraphenin content is 1.0 or more.
(2) The plant according to (1), wherein the glucoraphanin content is 20 mg/100 g (fresh weight) or more.
(3) The plant according to (1) or (2), wherein the glucoraphenin content is 50 mg/100 g (fresh weight) or less.
(4) An intergeneric crossbred plant from a Brassica plant and a Raphanus plant, which comprises a loss-of-function type glucoraphasatin synthase gene.
(5) The plant according to (4), wherein the loss-of-function type glucoraphasatin-synthesizing gene is the following gene (a) and/or (b):
   (a) a gene encoding a protein consisting of an amino acid sequence set forth in SEQ ID NO: 1 or an amino acid sequence having sequence identity 90% or more to the amino acid sequence, in which a constituent exon comprises a deletion of all or part of a 2-oxoglutarate-iron(II)-dependent oxygenase domain in the encoded protein; and/or
   (b) a gene comprising a nucleic acid sequence having sequence identity 70% to with a nucleic acid sequence set forth in SEQ ID NO: 2 or 3.
(6) The plant according to any one of (1) to (5), wherein the Brassica plant is Brassica oleracea.
(7) The plant according to any one of (1) to (6), wherein the Raphanus plant is Raphanus sativus.
(8) A plant according to any one of (1) to (7), which has a polyploidized chromosome.
(9) A method for creating a Brassicaceae intergeneric crossbred plant, the method comprising: a step of crossbreeding a first parent plant and a second parent plant; and a step of obtaining an intergeneric crossbred plant from the first parent plant and the second parent plant, wherein the first parent plant and the second parent plant are Brassicaceae plants of different genera, the first parent plant comprises 5 mg/100 g (fresh weight) or more of glucoraphanin, and the second parent plant comprises a loss-of-function type glucoraphasatin synthase gene.
(10) The method according to (9), wherein the loss-of-function type glucoraphasatin synthase gene is the following gene (a) and/or (b):
   (a) a gene encoding a protein consisting of an amino acid sequence set forth in SEQ ID NO: 1 or an amino acid sequence having sequence identity 90% or more to the amino acid sequence, in which a constituent exon comprises a deletion of all or part of a 2-oxoglutarate-iron(II)-dependent oxygenase domain in the encoded protein; and/or
   (b) a gene comprising a nucleic acid sequence having sequence identity 70% or more to the nucleic acid sequence set forth in SEQ ID NO: 2 or 3.
(11) The method according to (9) or (10), which further comprises a step of selecting an intergeneric crossbred plant comprising the loss-of-function type glucoraphasatin synthase gene from the intergeneric crossbred plant.
(12) The method according to any one of (9) to (11), wherein the first parent plant is a Brassica plant and the second parent plant is a Raphanus plant.
(13) The method according to any one of (9) to (12), wherein the Brassica plant is Brassica oleracea.
(14) The method according to any one of (9) to (13), wherein the Raphanus plant is Raphanus sativus.
(15) The method according to any one of (9) to (14), wherein the intergeneric crossbred plant comprises a polyploidized chromosome.
(16) A method for producing a Brassicaceae plant, the method comprising a step of cultivating the plant according to any one of (1) to (7).
(17) A food for which the plant according to any one of (1) to (7) is used as a raw material.
(18) A method for allowing a Brassicaceae plant to have a large amount of glucoraphanin, the method comprising: a first step of preparing a Brassicaceae plant comprising than 5 mg/100 g (fresh weight) or more of glucoraphanin as a first parent plant; a second step of preparing a Brassicaceae plant of a genus different from the first parent plant, in which the function of glucoraphasatin synthase is deficient or reduced, as a second parent plant; and a third step of crossbreeding the first parent plant and the second parent plant.
(19) The method according to (18), wherein the second step comprises modifying a glucoraphasatin synthase gene to cause the function of glucoraphasatin synthase to be deficient or reduced.

The present specification encompasses the contents described in the specification and/or drawings of Japanese Patent Application No. 2021-063196, on which the priority of the present application is based.

### Advantageous Effects of Invention

According to the present invention, a Brassicaceae plant comprising a large amount of glucoraphanin can be obtained.

### BRIEF DESCRIPTION OF DRAWING

FIG. 1 is a schematic diagram showing biosynthetic pathways of glucoraphanin and glucoraphenin in Brassicaceae plants.

FIG. 2 is a schematic diagram showing the structure of the wild-type GRS1 gene and the insertion positions of the retrotransposon in the nucleic acid sequences set forth in SEQ ID NOs: 2 and 3. FIG. 2(A) illustrates the structure of the wild-type GRS1 gene. FIG. 2(B) illustrates the structure of the nucleic acid sequence set forth in SEQ ID NO: 2. FIG. 2(C) illustrates the structure of the nucleic acid sequence set forth in SEQ ID NO: 3.

FIG. 3 is a graph showing the correlation of glucoraphanin contents in the leaves and seeds of individual Brassicaceae plants.

### DESCRIPTION OF EMBODIMENTS

As used herein, unless otherwise specified, chemical formulas depicted are meant to comprise all geometric and optical isomers. As used herein, the term “content” refers to a weight concentration (w/w) unless otherwise specified. As used herein, the term “derivative” refers to a compound that has been modified to such an extent that the skeletal structure of the compound is unaffected.

1. Intergeneric Crossbred Plant from Brassica Plant and Raphanus Plant

A first embodiment of the intergeneric crossbred plant from a Brassica plant and a Raphanus plant of the present invention (hereinafter also referred to as “the plant of the present invention”) is characterized in that the ratio of the glucoraphanin content to the glucoraphenin content is 1.0 or more. A second embodiment of the plant of the present invention is characterized by comprising the loss-of-function type glucoraphasatin synthase gene. The plant of the present invention may have one of or both of the characteristics of the first and second embodiments.

When the term “plant” is used herein simply, it encompasses any part of leaves, stems, flowers, buds, roots, and seeds unless otherwise specified or inconsistent. The plant of the present invention has the above-described characteristics, at least in any part of leaves, stems, flowers, buds, roots, and seeds.

As used herein, the term “glucoraphanin” refers to a compound represented by the following Formula (I) or a derivative or salt thereof.

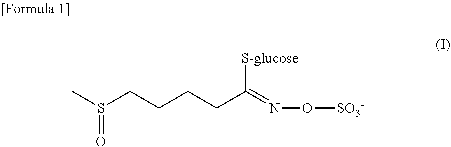

As used herein, the term “glucoraphenin” refers to a compound represented by the following Formula (II) or a derivative or salt thereof.

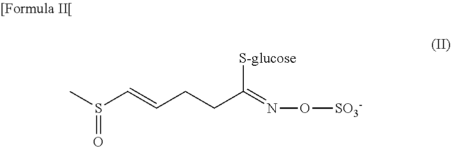

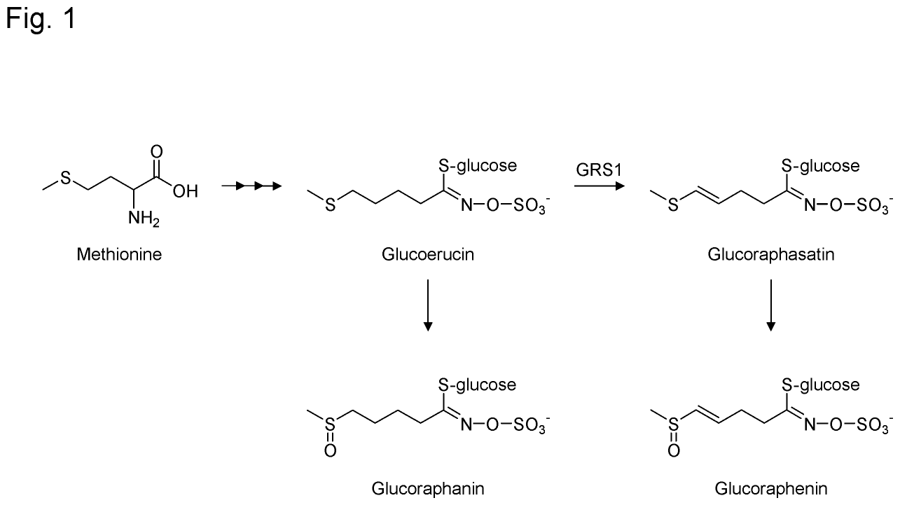

It has been clarified that glucoraphanin and glucoraphenin are biosynthesized by the biosynthetic pathways shown in FIG. 1 in Brassicaceae plants. Specifically, glucoerucin is synthesized by about 20 types of enzymatic reactions using methionine as a starting material. Glucoerucin is oxidized, thereby synthesizing glucoraphanin. In some Brassicaceae plants, glucoerucin is converted by the glucoraphasatin synthase (GRS1) gene to glucoraphasatin, and then glucoraphasatin is oxidized, thereby synthesizing glucoraphenin. Glucoraphasatin is converted to raphasatin, a pungent component, by the action of myrosinase, a hydrolase, and then to an odor component or a yellowing substance.

As used herein, “Brassicaceae plants” are classified into one family of the order Capparales of the subclass Dilleniidae of the class Dicotyledoneae of the phylum Angiospermae and include plants of many genera such as Cardamine, Arabis, Brassica, Rorippa, Raphanus, and Barbarea.

As used herein, “Brassica plants” are classified into one genus of the family Brassicaceae and include rapeseed, mizuna, Japanese pak choy (taisai), bok choy, Japanese mustard spinach (komatsuna), turnip, Chinese cabbage, cabbage, broccoli, leaf peony (habotan), mustard, kale, kohlrabi, cauliflower, and the like. Brassica plants used as parent plants of the plant of the present invention are preferably plants comprising relatively large amounts of glucoraphanin such as kale, broccoli, cabbage, kohlrabi, cauliflower, and the like. A preferred Brassica plant is Brassica oleracea. It is particularly preferable that the Brassica plant comprises 5 mg/100 g (fresh weight (FW)) or more or 10 mg/100 g FW or more of glucoraphanin. Such a plant of Brassica oleracea comprising a large amount of glucoraphanin may be obtained by, for example, the method described in Patent Literature 1.

As used herein, “Raphanus plants” are classified into one genus of the family Brassicaceae and include Japanese radish, Japanese wild radish, and the like. A preferred Raphanus plant used as a parent plant of the plant of the present invention is Raphanus sativus.

Raphanus plants, specifically Japanese radish, are characterized by comprising glucoraphasatin that is not synthesized in the related species of the same family Brassicaceae. The gene of glucoraphasatin synthase that converts glucoerucin to glucoraphasatin has been identified in Japanese radish. It has been revealed that this characteristic gene synthesizes glucoraphasatin.

As described in Patent Literature 1 and other literature, a mutant having a low glucoraphasatin content is present in Japanese radish in some cases. It is known that the structure of the glucoraphasatin synthase gene present at the end of the first linkage group of Japanese radish is altered from that of the wild type, and thus, the gene function is lost in this mutant. Since the normal glucoraphasatin synthase genotype is dominant, it was named the GRS1 (glucoraphasatin synthase 1) gene. The recessive genotype with the loss of function was named the grs1 gene. The amino acid sequence encoded by the GRS1 gene has the 2-oxoglutarate-iron(II)-dependent oxygenase domain representing an oxygenase that is universally present in plants.

The Raphanus plant used in the present invention is preferably a Raphanus plant comprising the loss-of-function type glucoraphasatin synthase (grs1) gene. grs1 may be comprised in the homozygous or heterozygous form in Raphanus plants. The Raphanus plant comprising the loss-of-function type glucoraphasatin synthase (grs1) gene can be selected using a method of DNA marker assay described in, for example, Patent Literature 4. Specifically, the polymerase chain reaction (PCR) method using DNA extracted from a sample plant as a template, a set of primers for specifically amplifying the GRS1 gene, and a set of primers for specifically amplifying the grs1 gene can be used.

As the Raphanus plant comprising the grs1 gene, a mutant may be selected by the above-described method from many progeny lines created by outcrossing and used. Alternatively, a wild-type Raphanus plant in which the GRS1 gene has been modified to cause the loss of function or reduce the function may be used. Any known method can be used as a method for modifying the GRS1 gene. For example, mutagenesis with insertion sequence transfer via a transposon, retrotransposon, plant virus, or the like can be mentioned. In addition, mutating treatments such as radiation irradiation treatment, heavy ion beam treatment, and treatment with a solution comprising a mutagen can be mentioned.

As used herein, the “intergeneric crossbred plant” refers to a crossbreed formed by crossbreeding between organisms classified in different genera, namely crossbred progeny. It is clearly distinguished from intergeneric crossbreeds, which are crossbreeds between different organisms within the same genus.

The plant of the present invention is a crossbred progeny created by crossbreeding of a Brassica plant and a Raphanus plant. In the first embodiment of the plant of the present invention, the ratio of the glucoraphanin content to the glucoraphenin content is 1.0 or more. In this embodiment, the amount of glucoraphanin comprised in the plant of the present invention is preferably 20 mg/100 g FW or more, particularly preferably 30 mg/100 g FW or more, 50 mg/100 g FW or more, 100 mg/100 g FW or more, or 150 mg/100 g FW or more. In addition, the amount of glucoraphenin comprised in the plant of the present invention is preferably 50 mg/100 g FW or less, particularly preferably 20 mg/100 g FW or less, 10 mg/100 g FW or less, 5 mg/100 g FW or less, 3 mg/100 g FW or less, or 2 mg/100 g FW or less.

The second embodiment of the plant of the present invention comprises the loss-of-function type glucoraphasatin synthase gene. In this embodiment, the plant of the present invention is created by using, as a parent plant, a Raphanus plant carrying the loss-of-function type glucoraphasatin-synthesizing gene (grs1 gene) in the homozygous or heterozygous form. In a case in which a Raphanus plant carrying the grs1 gene in the heterozygous form is used, half of the intergeneric crossbred plants become the loss-of-function type. Therefore, the plant of the present invention can be obtained by selecting the loss-of-function type progeny. As the method for selecting loss-of-function type plants, for example, the method for determining DNA marker assay described in Patent Literature 4 can be used. Specifically, the PCR method using DNA extracted from a sample plant as a template, a set of primers for specifically amplifying the GRS1 gene and a set of primers for specifically amplifying the grs1 gene can be used.

In this embodiment, the grs1 gene is not particularly limited as long as it has a structure in which the function of GRS1 is lost. However, it is preferable that the grs1 gene comprises the following nucleic acid sequence (a) and/or (b):
(a) a nucleic acid sequence encoding a protein consisting of an amino acid sequence set forth in SEQ ID NO: 1 or an amino acid sequence having sequence identity 90% or more to the amino acid sequence, in which a constituent exon comprises a deletion of all or part of a 2-oxoglutarate-iron(II)-dependent oxygenase domain in the encoded protein; and/or
(b) a nucleic acid sequence comprising a nucleic acid sequence having sequence identity 70% or more to a nucleic acid sequence set forth in SEQ ID NO: 2 or 3.

It is preferable that the grs1 gene comprises: a nucleic acid sequence encoding a protein consisting of an amino acid sequence set forth in SEQ ID NO: 1 or an amino acid sequence having sequence identity 91% or more, 92% or more, 93% or more, 94% or more, 95% or more, 96% or more, 97% or more, 98% or more, or 99% or more to the amino acid sequence, in which a constituent exon comprises a deletion of all or part of a 2-oxoglutarate-iron(II)-dependent oxygenase domain in the encoded protein; and/or a nucleic acid sequence having sequence identity 75% or more, 80% or more, 85% or more, 90% or more, or 95% or more to a nucleic acid sequence set forth in SEQ ID NO: 2 or 3.

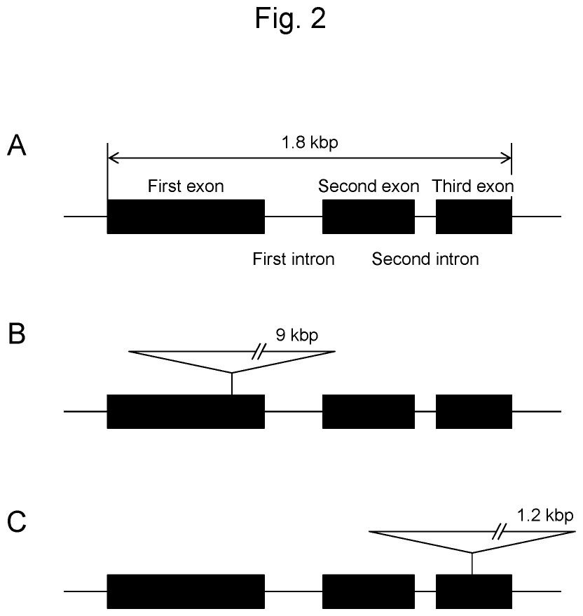

The nucleic acid sequences set forth in SEQ ID NOS: 2 and 3 are examples of the nucleic acid sequence of the grs1 gene. Specifically, each of the nucleic acid sequences set forth in SEQ ID NOS: 2 and 3 has a structure in which a retrotransposon is inserted into the exon sequence of the GRS1 gene. FIG. 2 illustrates the structure of the wild-type GRS1 gene and the insertion positions of the retrotransposon in the nucleic acid sequences set forth in SEQ ID NOs: 2 and 3. FIG. 2(A) illustrates the structure of the wild-type GRS1 gene. The GRS1 gene has three exons (first, second, and third exons) and two introns (first and second introns). FIG. 2(B) illustrates the structure of the nucleic acid sequence set forth in SEQ ID NO: 2. In this sequence, a retrotransposon of about 9 kbp is inserted into the first exon of the GRS1 gene. FIG. 2(C) illustrates the structure of the nucleic acid sequence set forth in SEQ ID NO: 3. In this sequence, a retrotransposon of about 1.2 kbp is inserted into the third exon of the GRS1 gene.

The plant of the present invention can comprise glucoraphanin at a high concentration by comprising a loss-of-function type glucoraphasatin synthase gene. It is presumed that in a Raphanus plant (especially Japanese radish) comprising a loss-of-function type glucoraphasatin synthase gene in the homozygous form, conversion from glucoerucin to glucoraphasatin is inhibited, resulting in the accumulation of glucoerucin. Although the leaves of Japanese radish do not have a function to convert glucoerucin to glucoraphanin efficiently, a Brassica plant (especially Brassica oleracea) has a function to biosynthesize glucoraphanin from glucoerucin. It can be said that the intergeneric crossbred plant of the present invention can compris glucoraphanin at a high concentration because it suppresses conversion from glucoerucin to glucoraphasatin while maintaining glucoerucin-to-glucoraphanin conversion capacity from a Brassica plant, thereby promoting conversion from glucoerucin to glucoraphanin.

The plant of the present invention may be a diploid but may also be an alloploid, for example, a triploid or tetraploid. Such an alloploid of the plant of the present invention can restore or improve fertility. Such an alloploid may be created by any known method. It can be created by, for example, colchicine treatment (see, for example, Cho et al., Breeding Science, Vol. 3, pp. 31-41 (2001) and Ogasawara et al., Horticultural research (Japan), Vol. 11, No. 2, pp. 189-194 (2012)).

The plant of the present invention can be used as a food or feed material. As a food material, the plant of the present invention can be used as a usual vegetable or in the form of a liquid (drink), a powder, granules, or the like. Alternatively, it can be used as a material for extracting or purifying glucoraphanin. Purified glucoraphanin can be used in, for example, dietary supplements, pharmaceuticals, and the like.

### 2. Method for Creating Brassicaceae Intergeneric Crossbred Plant

The method for creating the Brassicaceae intergeneric crossbred plant of the present invention (hereinafter also referred to as “the creation method of the present invention”) is characterized by comprising: a step of crossbreeding a first parent plant and a second parent plant: and a step of obtaining an intergeneric crossbred plant from the first parent plant and the second parent plant, wherein the first parent plant and the second parent plant are Brassicaceae plants of different genera, the first parent plant comprises 5 mg/100 g FW or more of glucoraphanin, and the second parent plant comprises a loss-of-function type glucoraphasatin synthase gene.

In the creation method of the present invention, the “first parent plant” is a Brassicaceae plant comprising 5 mg/100 g FW or more, preferably 10 mg/100 g FW or more of glucoraphanin, which is classified into a genus different from the second parent plant. The first parent plant is not particularly limited as long as it is a Brassicaceae plant classified into a genus different from the second parent plant: however, it is preferably a Brassica plant, particularly preferably a plant comprising a relatively large amount of glucoraphanin, such as kale, broccoli, cabbage, kohlrabi, or cauliflower. It is preferably Brassica oleracea. Such a plant of Brassica oleracea comprising a large amount of glucoraphanin may be obtained by, for example, the method described in Patent Literature 1.

In the creation method of the present invention, the “second parent plant” is a Brassicaceae plant comprising a loss-of-function type glucoraphasatin synthase gene, which is classified into a genus different from the first parent plant. The second parent plant is not particularly limited as long as it is a Brassicaceae plant classified into a genus different from the first parent plant: however, it is preferably a Raphanus plant. It is preferably Raphanus sativus.

The loss-of-function type glucoraphasatin synthase (grs1) gene may be comprised in the homozygous or heterozygous form in the second parent plant. As the method for selecting the second parent plant comprising the grs1 gene, for example, the method described in Patent Literature 4 can be used. Specifically, the PCR method using DNA extracted from a sample plant as a template, a set of primers for specifically amplifying the GRS1 gene, and a set of primers for specifically amplifying the grs1 gene can be used.

As the second parent plant comprising the grs1 gene, a mutant may be selected by the above-described method from many progeny lines created by outcrossing and used. Alternatively, a plant in which the wild-type GRS1 gene has been modified to cause the loss of function or reduce the function may be used. Any known method can be used as a method for modifying the GRS1 gene. For example, mutagenesis with insertion sequence transfer via a transposon, retrotransposon, plant virus, or the like can be included. In addition, mutation treatments such as radiation irradiation treatment, heavy ion beam treatment, and treatment with a solution comprising a mutagen can be included.

The grs1 gene comprised in the second parent plant is not particularly limited as long as it has a structure in which the function of GRS1 is lost. It is preferable that the grs1 gene comprises the following nucleic acid sequence (a) and/or (b):
(a) a nucleic acid sequence encoding a protein consisting of an amino acid sequence set forth in SEQ ID NO: 1 or an amino acid sequence having sequence identity 90% or more to the amino acid sequence, in which a constituent exon comprises a deletion of all or part of a 2-oxoglutarate-iron(II)-dependent oxygenase domain in the encoded protein: and/or
(b) a nucleic acid sequence comprising a nucleic acid sequence having sequence identity 70% or more to a nucleic acid sequence set forth in SEQ ID NO: 2 or 3.

It is preferable that the grs1 gene comprises: a nucleic acid sequence encoding a protein consisting of an amino acid sequence set forth in SEQ ID NO: 1 or an amino acid sequence having sequence identity 91% or more, 92% or more, 93% or more, 94% or more, 95% or more, 96% or more, 97% or more, 98% or more, or 99% or more to the amino acid sequence, in which a constituent exon comprises a deletion of all or part of a 2-oxoglutarate-iron(II)-dependent oxygenase domain in the encoded protein: and/or a nucleic acid sequence having sequence identity 75% or more, 80% or more, 85% or more, 90% or more, or 95% or more to a nucleic acid sequence set forth in SEQ ID NO: 2 or 3.

In a case in which a plant carrying the grs1 gene in the heterozygous form plant is used as the second parent plant, half of the intergeneric crossbred plants become the loss-of-function type. In this case, it is preferable that the creation method of the present invention comprises a step of selecting loss-of-function type plants. As the method for selecting loss-of-function type plants, for example, the method described in Patent Literature 4 can be used. Specifically, the PCR method using DNA extracted from a sample plant as a template, a set of primers for specifically amplifying the GRS1 gene, and a set of primers for specifically amplifying the grs1 gene can be used.

In the creation method of the present invention, the first parent plant and the second parent plant are classified into different genera and an intergeneric crossbred plant is obtained by crossbreeding. The crossbreeding used herein is not particularly limited. A crossbreeding technique used in general for the improvement of cultivars or the like can be used. In addition, plants obtained by crossbreeding may be further subjected to self-mating for several generations, backcrossing for several generations, or repeating self-mating and backcrossing as appropriate.

The creation method of the present invention may be a method for creating a diploid or a method for creating an alloploid such as a triploid or tetraploid.

Detailed conditions such as parent plants used in the creation method of the present invention, detailed properties of the created plants, and the like are the same as the conditions, properties, and the like described in “1. Intergeneric Crossbred Plant from Brassica Plant and Raphanus Plant,” unless otherwise specified in this section, and unless there is a particular contradiction.

### 3. Method for Producing Brassicaceae Plant

The method for producing a Brassicaceae plant of the present invention (hereinafter also referred to as “the production method of the present invention”) is characterized by comprising a step of cultivating the plant of the present invention described in “1. Intergeneric Crossbred Plant from Brassica Plant and Raphanus Plant.” According to the production method of the present invention, a plant comprising a large amount of glucoraphanin can be obtained.

A plant produced by the production method of the present invention is an intergeneric crossbred plant from a Brassica plant and a Raphanus plant. The plant produced by the production method of the present invention is preferably a dietary plant or a plant for feed use, more preferably a dietary vegetable. The plant produced by the production method of the present invention is preferably a crossbreed of Brassica oleracea and Raphanus sativus, namely Raphanobrasica. According to the above preferred embodiment, a plant from which humans or animals (e.g., mammals such as dogs, cats, bovines, horses, pigs, sheep, monkeys, and ferrets, and birds such as chickens) can ingest a large amount of Raphanobrasica can be obtained.

The plant cultivated by the production method of the present invention may be an intergeneric crossbred plant of the F1 generation obtained by crossbreeding a Brassica plant and a Raphanus plant or an intergeneric crossbred plant of the F2 generation obtained by self-mating of the F1 generation. It may further be an intergeneric crossbred plant obtained by repeating self-mating of the F2 and subsequent generations. Alternatively, it may be an intergeneric crossbred plant obtained by backcrossing the F2 and subsequent generations.

### 4. Food

The food in the present invention is characterized in that the plant of the present invention is used as a raw material as described in “1. Intergeneric Crossbred Plant from Brassica Plant and Raphanus Plant.” As used herein, “food” refers to a substance or composition in a form suitable for being ingested by a human. The food in the present invention may be a plant per se as a vegetable or a dish for which the vegetable is used. The food may be, for example, in the form of a liquid (drink), powder, granules, or the like processed from the plant. Alternatively, it may be a drink or a nutritional supplement (supplement) such as a powder, granules, tablets, capsules, or the like comprising glucoraphanin extracted and purified from the plant.

### 5. Method for Allowing Brassicaceae Plant to Comprise Large Amount of Glucoraphanin

The method for allowing a Brassicaceae plant to comprise a large amount of glucoraphanin of the present invention (hereinafter also referred to as “the amount increasing method of the present invention”) is characterized by comprising: a first step of preparing a Brassicaceae plant comprising 5 mg/100 g FW or more of glucoraphanin as a first parent plant: a second step of preparing a Brassicaceae plant of a genus different from the first parent plant, in which the function of glucoraphasatin synthase is deficient or reduced, as a second parent plant; and a third step of crossbreeding the first parent plant and the second parent plant.

In the amount increasing method of the present invention, the first step is a step of preparing a Brassicaceae plant comprising than 5 mg/100 g FW or more, preferably 10 mg/100 g FW or more of glucoraphanin as a first parent plant. The “first parent plant” used herein is not particularly limited as long as it is a Brassicaceae plant classified into a genus different from the second parent plant; however, it is preferably a Brassica plant, particularly preferably a plant comprising a relatively large amount of glucoraphanin, such as kale, broccoli, cabbage, kohlrabi, or cauliflower. It is preferably Brassica oleracea.

The method for creating a plant comprising 5 mg/100 g FW or more of glucoraphanin is not particularly limited. For example, it may be the method described in Patent Literature 1. Alternatively, for example, the following method can be used. Many Brassica plant progeny lines are produced by outcrossing. Among the obtained progeny lines, one or more lines comprising glucoraphanin at a high concentration are selected, self-mating or outcrossing is performed, and if necessary, the crossing is repeated, thereby obtaining a line of Brassica plants comprising a large amount of glucoraphanin.

In the amount increasing method of the present invention, the second step is a step of preparing a Brassicaceae plant of a genus different from the first parent plant, in which the function of glucoraphasatin synthase is deficient or reduced, as a second parent plant. The “second parent plant” used herein is not particularly limited as long as it is a Brassicaceae plant classified into a genus different from the first parent plant. It is preferably a Raphanus plant. It is preferably Raphanus sativus.

The second parent plant is a plant in which the function of glucoraphasatin synthase (GRS1) is deficient or reduced. More specifically, it is a plant comprising the loss-of-function type glucoraphasatin synthase (grs1) gene. The grs1 gene may be comprised in the homozygous or heterozygous form. As the method for selecting the second parent plant comprising the grs1 gene, for example, the method described in Patent Literature 4 can be used. Specifically, the PCR method using DNA extracted from a sample plant as a template, a set of primers for specifically amplifying the GRS1 gene, and a set of primers for specifically amplifying the grs1 gene can be used.

As the second parent plant comprising the grs1 gene, a mutant may be selected by the above-described method from many progeny lines created by outcrossing and used. Alternatively, a plant in which the wild-type GRS1 gene has been modified to cause the loss of function or reduce the function may be used. Any known method can be used as a method for modifying the GRS1 gene. For example, mutagenesis with insertion sequence transfer via a transposon, retrotransposon, plant virus, or the like can be included. In addition, mutation treatments such as radiation irradiation treatment, heavy ion beam treatment, and treatment with a solution comprising a mutagen can be included.

The third step of the amount increasing method of the present invention is a step of crossbreeding the first parent plant and the second parent plant. The crossbreeding is not particularly limited. A crossbreeding technique used in general for the improvement of cultivars or the like can be used. In addition, plants obtained by crossbreeding may be further subjected to self-mating for several generations, backcrossing for several generations, or repeating self-mating and backcrossing as appropriate.

Since the amount increasing method of the present invention comprises the steps described above, Brassicaceae plants are allowed to comprise a large amount of glucoraphanin. Brassicaceae plants obtained by the amount increasing method of the present invention comprise preferably 20 mg/100 g FW or more, 30 mg/100 g FW or more, 50 mg/100 g FW or more, 100 mg/100 g FW or more, or 150 mg/100 g FW or more of glucoraphanin.

Detailed conditions such as parent plants used in the amount increasing method of the present invention, detailed properties of the created plants, and the like are the same as the conditions, properties, and the like described in “1. Intergeneric Crossbred Plant from Brassica Plant and Raphanus Plant” and “2. Method for Creating Brassicaceae Plant,” unless otherwise specified in this section, and unless there is a particular contradiction.

### Examples

Hereinafter, the present invention is described more specifically with reference to Examples. However, the scope of the present invention is not intended to be limited by these Examples.

### [Example 1] Creation of Intergeneric Crossbred Plant (Raphanobrasica)

An intergeneric crossbred plant between a Raphanus plant and a Brassicaceae plant was created by the following procedure. As a Brassicaceae plant, a kale line “KK-45” comprising glucoraphanin was used. As a Raphanus plant, a commercially available Japanese radish cultivar “Nishimachi-Risou” was used. It is known that individuals carrying the functional type GRS1 gene in the heterozygous form and individuals carrying the loss-of-function type GRS1 gene in the heterozygous form coexist within the cultivar (see Patent Literature 4). A DNA marker test was carried out for Nishimachi-Risou in advance, and individuals carrying the gene in the heterozygous form (hereinafter also referred to as “AKO”) were selected at the seedling stage. Four individuals (AKO103, AKO108, AKO110, and AKO118) were crossbred as seed parents. The cultivars/lines used herein and the intergeneric crossbreeds created are listed in Table 1.

TABLE 1

| Cultivar/ | Plant | |
|---|---|---|
| Line Name | species | Remarks |
| Nishimachi- | Japanese | Commercially available Japanese radish cultivar |
| Risou | radish | |
| KK-45 | Kale | Kale line obtained by selection and self-mating by the present inventors |
| AKO103 | Japanese | Individual carrying GRS1gene in the heterozygous form of functional type/ |
| | radish | loss-of-function type selected from Nishimachi-Risou |
| AKO108 | Japanese | Individual carrying GRS1gene in the heterozygous form of functional type/ |
| | radish | loss-of-function type selected from Nishimachi-Risou |
| AKO110 | Japanese | Individual carrying GRS1gene in the heterozygous form of functional type/ |
| | radish | loss-of-function type selected from Nishimachi-Risou |
| AKO118 | Japanese | Individual carrying GRS1gene in the heterozygous form of functional type/ |
| | radish | loss-of-function type selected from Nishimachi-Risou |
| AKO103 × | Rafanobrasica | Crossbred progeny of AKO103 and KK45 |
| KK-45 | | |
| AKO108 × | Rafanobrasica | Crossbred progeny of AKO108 and KK45 |
| KK-45 | | |
| AKO110 × | Rafanobrasica | Crossbred progeny of AKO110 and KK45 |
| KK-45 | | |
| AKO118 × | Rafanobrasica | Crossbred progeny of AKO118 and KK45 |
| KK-45 | | |

DNA marker assay of Nishimachi-Risou and DNA marker assay of each crossbred progeny were carried out by the following procedure. DNA was extracted from the leaves of Nishimachi-Risou, and a PCR reaction was performed using a primer set consisting of three primers listed in Table 2. A “loss-of-function type” gene was determined to be present when a 392-bp DNA amplification was observed. A “functional type” gene was determined to be present when a 222-bp DNA amplification was observed.

TABLE 2

| | | SEQ |
|---|---|---|
| | | ID |
| Primer type | Primer sequence | NO |
| Primer for | 5′-TCCAGGTTGGGATAGCTTGT-3′ | 4 |
| detecting | | |
| loss-of- | | |
| function | | |
| type gene | | |
| (reverse) | | |
| Primer for | 5′-TGAAACCTTACCCCAAAACG-3′ | 5 |
| detecting | | |
| functional | | |
| type gene | | |
| (reverse) | | |
| Common | 5′-GCAGGAGAGGATGCTTGAAGG- | 6 |
| primer | 3′ | |
| (forward) | | |

Nishimachi-Risou individuals in which both 392-bp and 222-bp DNA amplifications were observed were determined to be “heterozygous form” and Nishimachi-Risou individuals in which only 222-bp DNA amplification was observed were determined to be “wild-type.” DNA marker assay of each crossbred progeny was performed by the following procedure. Individuals of the crossbred progeny carrying the functional type GRS1 gene were determined to be “functional type” crossbred progeny and individuals of the crossbred progeny carrying the loss-of-function type GRS1 (grs1) gene were determined to be “loss-of-function type” crossbred progeny.

### [Example 2] Determination of Contents of Various Glucosinolates in Leaves

Samples used for analysis of the glucosinolate content were prepared as follows. Three true leaves having a leaf length of 20 cm were collected from each line in the field. A 10-cm tip of the leaf blade was taken from each leaf and the vein running down the middle of the leaf was removed. The leaves were lyophilized for 4 to 5 days (Freeze Dryer manufactured by LABCONCO) and the resulting dried samples were crushed (Multi-Beads Shocker manufactured by Yasui Kikai Corporation). Then, 0.1 g of dry powder was precisely weighed, 5 mL of 80% methanol was added, and the mixture was shaken and stirred at room temperature for 30 minutes. After centrifugation at 3000 rpm for 10 minutes, the supernatant was used as the glucosinolate extract. The glucosinolate extract was adsorbed on a DEAE-Sepharose column and desulfurized with acid sulfatase (25° C. for 18 hours). The desulfurized glucosinolate was eluted with ion exchange water, thereby obtaining a desulfo-glucosinolate solution. The desulfo-glucosinolate solution was subjected to HPLC under the following conditions, and a chromatogram was obtained at a UV detection wavelength of 229 nm.
Equipment used: LC-20A, Shimadzu Corp., Japan
Column type: COSMOSIL 5C18-II, 150×4.6 mm, Nacalai-Tesque Inc., Japan
Mobile phase solvent composition: 20% acetonitrile
Sample injection volume: 20 μL
Flow rate: 1.5 mL/min
Column temperature: 30° C.

The content of each glucosinolate (glucoraphanin, glucoraphenin, glucoerucin, or glucoraphasatin) in each sample was calculated based on the results of subjecting the corresponding authentic glucosinolate preparation having a known concentration to HPLC under the same conditions. Table 3 shows the measurement results of each glucosinolate for each cross combination.

TABLE 3

| Cross | GRS1 | Number of | Average glucosinolate content (mg/100 g FW) | | | |
|---|---|---|---|---|---|---|
| combination | genotype | individuals | Glucoraphanin | Glucoraphenin | Glucoerucin | Glucoraphasatin |
| AKO103 × | Functional | 18 | 97.3 | 186.9 | 8.3 | 31.0 |
| KK-45 | type | | | | | |
| | Loss-of- | 29 | 249.1 | 0.3 | 61.2 | 0.1 |
| | function type | | | | | |
| AKO110 × | Functional | 30 | 113.9 | 230.7 | 8.5 | 30.2 |
| KK-45 | type | | | | | |
| | Loss-of- | 33 | 237.1 | 1.6 | 57.1 | n.d. |
| | function type | | | | | |
| AKO118 × | Functional | 45 | 112.4 | 172.2 | 4.0 | 13.9 |
| KK-45 | type | | | | | |
| | Loss-of- | 38 | 221.9 | 1.5 | 31.3 | n.d. |
| | function type | | | | | |
| AKO108 × | Functional | 29 | 63.0 | 130.3 | 5.9 | 23.1 |
| KK-45 | type | | | | | |
| | Loss-of- | 25 | 138.1 | 1.1 | 37.6 | n.d. |
| | function type | | | | | |
| AKO103 | Loss-of- | 3 | 54.7 | n.d. | 92.0 | n.d. |
| | function type | | | | | |
| | (homozygous | | | | | |
| | progeny of | | | | | |
| | self-mating) | | | | | |
| KK-45 | Non-carrier | 8 | 123.2 | n.d. | n.d. | n.d. |

n.d.: Not detected

Within all cross combinations, the average glucoraphanin content of the population having the loss-of-function type gene was higher than the population carrying the functional-type gene. The ratio of loss-of-function type glucoraphanin content/functional type glucoraphanin content is in a range of 1.97 to 2.56. The loss-of-function type individuals comprised no or very little glucoraphenin and glucoraphasatin. These results revealed that suppressing the expression of the GRS1 gene in Rafanobrasica causes most of the glucoraphanin to be metabolized to glucoraphanin, resulting in an increased glucoraphanin content.

### [Example 3] Determination of Contents of Various Glucosinolates in Roots, Buds, and Stems

Root, bud, and stem samples were prepared for analyzing the glucosinolate content by the following procedures follows. Ten individuals of each line were dug up with the roots from the field and washed with water. Roots were cut into 0.5- to 1-cm thick slices across a position about 5 cm below the stem-hypocotyl boundary. As for the buds, terminal flower buds were collected from ten bolting individuals of each line. Stems about 10 cm below the boundary with the bud were collected from the ten bolting individuals of each line. Each sample was lyophilized for 4 to 5 days and the dried sample was crushed. Thereafter, extraction, desulfurization, and HPLC measurement were carried out by the same procedure as in Example 2. Table 4 shows the measurement results of each glucosinolate for each sample.

TABLE 4

| Measurement | Cross | GRS1 | Number of | Average glucosinolate content (mg/100 g FW) | | | |
|---|---|---|---|---|---|---|---|
| site | combination | genotype | individuals | Glucoraphanin | Glucoraphenin | Glucoerucin | Glucoraphasatin |
| Root | AKO103 × | Functional | 10 | 11.4 | 32.6 | 127.8 | 345.9 |
| | KK-45 | type | | | | | |
| | | Loss-of- | 10 | 22.1 | n.d. | 295.6 | 2.8 |
| | | function type | | | | | |
| But | AKO110 × | Functional | 10 | 180.1 | 405.6 | 19.6 | 42.1 |
| | KK-45 | type | | | | | |
| | | Loss-of- | 10 | 432.8 | 1.8 | 65.6 | n.d. |
| | | function type | | | | | |
| Step | AKO110 × | Functional | 10 | 78.0 | 164.6 | 22.4 | 58.7 |
| | KK-45 | type | | | | | |
| | | Loss-of- | 10 | 176.4 | 0.3 | 56.7 | n.d. |
| | | function type | | | | | |

n.d.: Note detected

As shown in Table 4, the average glucoraphanin content was higher in the loss-of-function type individuals than in the functional type individuals at all sites. In addition, the loss-of-function type individuals comprised no or very little glucoraphenin and glucoraphasatin. These results revealed that suppressing the expression of the GRS1 gene in Raphanobrasica causes the glucoraphanin content to increase regardless of the collection sites.

[Example 4] Correlation between Glucoraphanin Contents in Seeds and Leaves of Brassicaceous Vegetables

Seed and leaf samples of Brassicaceae vegetables were prepared for analyzing the glucoraphanin content by the following procedure. KK-45 and commercially available kale cultivars, which were 68 lines in total, were used as Brassicaceae vegetables. Three true leaves having a leaf length of 20 cm were collected for one individual of each line from a greenhouse. A 10-cm tip of the leaf blade was taken from each leaf and the vein running down the middle of the leaf was removed. Seeds were collected from the same individual from which the leaves were collected, and 0.5 g of seeds per individual were used. Each sample was lyophilized for 4 to 5 days and the dried sample was crushed. Thereafter, extraction, desulfurization, and HPLC measurement were carried out by the same procedure as in Example 2. The HPLC measurement conditions were as follows.
Equipment used: LC-20A, Shimadzu Corp., Japan
Column type: COSMOSIL 5C18-II, 150×4.6 mm, Nacalai-Tesque Inc., Japan
Mobile phase solvent composition: 20% acetonitrile
Sample injection volume: 20 μL
Flow rate: 1.5 mL/min
Column temperature: 30° C.

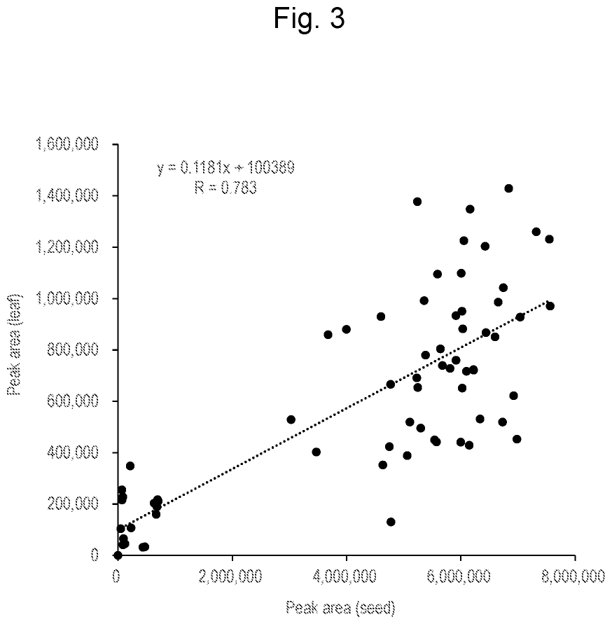

FIG. 3 shows the correlation between HPLC peak areas (contents) of glucoraphanin in samples from the leaves and seeds of each individual.

As shown in FIG. 3, there was a correlation between the HPLC peak areas of glucoraphanin in samples from the leaves and seeds of Brassicaceae vegetables, and cultivars with high glucoraphanin contents in leaves also had high glucoraphanin contents in their seeds. These results suggested that among Brassicaceae intergeneric crossbred plants, individuals with high leaf glucoraphanin contents also have high seed glucosinolate contents.

### Industrial Applicability

The present invention is an invention that can be used in the agricultural industry. the food manufacturing industry. the pharmaceutical manufacturing industry. and the like.

All publications. patents. and patent applications cited herein are incorporated herein by reference in their entirety.

1. An intergeneric crossbred plant from a Brassica plant and a Raphanus plant, for which a ratio of glucoraphanin content to glucoraphenin content is 1.0 or more.

2. The plant according to claim 1, wherein the glucoraphanin content is 20 mg/100 g (fresh weight) or more.

3. The plant according to claim 1, wherein the glucoraphenin content is 50 mg/100 g (fresh weight) or less.

4. An intergeneric crossbred plant from a Brassica plant and a Raphanus plant, which comprises a loss-of-function type glucoraphasatin synthase gene.

5. The plant according to claim 4, wherein the loss-of-function type glucoraphasatin-synthesizing gene is the following gene (a) and/or (b):
(a) a gene encoding a protein consisting of an amino acid sequence set forth in SEQ ID NO: 1 or an amino acid sequence having sequence identity 90% or more to the amino acid sequence, in which a constituent exon comprises a deletion of all or part of a 2-oxoglutarate-iron(II)-dependent oxygenase domain in the encoded protein; and/or
(b) a gene comprising a nucleic acid sequence having sequence identity 70% or more to a nucleic acid sequence set forth in SEQ ID NO: 2 or 3.

6. The plant according to claim 1, wherein the Brassica plant is Brassica oleracea.

7. The plant according to claim 1, wherein the Raphanus plant is Raphanus sativus.

8. A plant according to claim 1, which has a polyploidized chromosome.

9. A method for creating a Brassicaceae intergeneric crossbred plant, the method comprising:
a step of crossbreeding a first parent plant and a second parent plant; and
a step of obtaining an intergeneric crossbred plant from the first parent plant and the second parent plant, wherein
the first parent plant and the second parent plant are Brassicaceae plants of different genera,
the first parent plant comprises 5 mg/100 g (fresh weight) or more of glucoraphanin, and the second parent plant comprises a loss-of-function type glucoraphasatin synthase gene.

10. The method according to claim 9, wherein the loss-of-function type glucoraphasatin synthase gene is the following gene (a) and/or (b):
(a) a gene encoding a protein consisting of an amino acid sequence set forth in SEQ ID NO: 1 or an amino acid sequence having sequence identity 90% or more the amino acid sequence, in which a constituent exon comprises a deletion of all or part of a 2-oxoglutarate-iron(II)-dependent oxygenase domain in the encoded protein; and/or
(b) a gene comprising a nucleic acid sequence having sequence identity 70% or more to a nucleic acid sequence set forth in SEQ ID NO: 2 or 3.

11. The method according to claim 9, which further comprises a step of selecting an intergeneric crossbred plant comprising the loss-of-function type glucoraphasatin synthase gene from the intergeneric crossbred plant.

12. The method according to claim 9, wherein the first parent plant is a Brassica plant and the second parent plant is a Raphanus plant.

13. The method according to claim 9, wherein the Brassica plant is Brassica oleracea.

14. The method according to claim 9, wherein the Raphanus plant is Raphanus sativus.

15. The method according to claim 9, wherein the intergeneric crossbred plant comprises a polyploidized chromosome.

16. A method for producing a Brassicaceae plant, the method comprising a step of cultivating the plant according to claim 1.

17. A food for which the plant according to claim 1 is used as a raw material.

18. A method for allowing a Brassicaceae plant to have a large amount of glucoraphanin, the method comprising:
a first step of preparing a Brassicaceae plant comprising 5 mg/100 g (fresh weight) or more of glucoraphanin as a first parent plant;
a second step of preparing a Brassicaceae plant of a genus different from the first parent plant, in which the function of glucoraphasatin synthase is deficient or reduced, as a second parent plant; and
a third step of crossbreeding the first parent plant and the second parent plant.

19. The method according to claim 18, wherein the second step comprises modifying a glucoraphasatin synthase gene to cause the function of glucoraphasatin synthase to be deficient or reduced.

20. The plant according to claim 1, for which a ratio of glucoraphanin content to glucoerucin is 1.0 or more.

21. The plant according to claim 1, which is a part of a plant except for a root


SEQUENCE LISTING (6 sequences)

SEQ ID NO: 1 - protein, 372 aa, Raphanus sativus
MSSNVVSGVEMDGSNERKVFDDKKMGVKALADAGIKELPAMFRAPPSILESLKAARASQDANLFPTIDLKGVSLHYKDQDLMTRRNVVEQIRDASAKWGFFRVTNHGFSKDLQERMLEGLRRFHAQDPSVKRQYYTRDHTRNFLYYTNVDLFTSDSASWRDTTICYTAPDRPRPEDLPAVLGEVILEYSKEMTSLGELIFELLSEALGLDTNYFKDLDCVKSQMMLGQYYPPCPQPDLTLGLSKHSDFSFLTVLLQDNIGGLQVLHDDAWFDVPPVPGCFVVNIGDLLQFITNDMFISAEHRVLAHTASVPRVSVPYFFTTFKKVNPRVYGPIKELLSEDNPRKYRDCSMTEISEIFSSNEITIPRLHQLRI

SEQ ID NO: 2 - DNA, 10555 bp, Raphanus sativus
atagcattagaaaagcttttaaagaaacaatgtccagcaacgtcgttagtggggtcgaaatggatggttcgaatgaaagaaaagttttcgacgataagaaaatgggcgtaaaagccctggcggatgcaggaatcaaagagctcccagccatgttccgtgcacctccgagtattttagaaagcctgaaagcagcacgagcttctcaggatgcgaacctcttcccgaccattgatctgaaaggagtgagcctgcactacaaggatcaagatttgatgacgcggcggaacgtggtggagcagatcagagatgcgtctgcgaaatggggtttcttccgagtgaccaatcacgggttctcgaaggatttgcaggagaggatgcttgaagggcttcgtcggtttcacgcgcaagatccatcagttaagagacagtactacacacgtgatcacactcggaactttctttactacaccaacgtcgatctcttcacctctgattccgccagttggagagacacaactatttgttacacagccccggatcgtccccgacctgaggattgaaagttcagggacaataagttaataaggaacgcacagaacttccaagaacacttccttgattaatcagaaaccttttaaacaatcttcctagatctgtttaatccgatttatactcagtcacacatcgactagagacggaccagtttactaatcttgtcactcgacaataacacaggacaagctatcccaacctggatactctcctattagactgcttctcaagaacactcttcttgcttaagcacgtcaaaacactaaaaaccgtaacctaaatctacccaaggtaatttggttatatacacctctcacaatatctcccctgagatatcgtgatatatacaaatccccatcgttattcatctctccgagatatcaggttcaaacgattgcctcgtcgccacgtcatcattcagctactttctcgcgtcaccatgtgttacacaacgctccatatgtgtaactcactaatcttgttccttgtctcacaagcactaatccttggtgctttcattctccccctttttatctgaatgtgtcaccctaagcaacctgcacattcaaaatgataacacaccaaaccatatccgcacaaaagtgataaacaagatgagaggagaaaaacatagattagtctatcagaacagtgccacgcacatgaaggttcatacatcattctgcctcacatctaatccgcaaataacacactagatgactagaccacgacctatcataacacataagagactaggacaatgtgaactcagccactagaacgaatcactctcatcagattcttctcttcctccattttcttcatcatcctcacgcggaccatattctccccctgcttaggtgcacatacagataaaacacttagatcaaacaacataagtcatagatcacaggaaaagaagtgatactcttactcctcagacgaacacgaacgttctttagatctgtaatgagacgatcaatgtcttctgcaagatcaggaagactagagttagctcctgttcctctccctgcctttctgtctttgaccactgctttgggatagcccatttcttcacaatcattaggatccggtggtatgacacgctgactcataagcacttgctgaatgagagtaggataggtgattctgcgagactccttagtcagaaccttctttgccatggagagaatctgctggtagactatcttcccaaaatcaaaacctcggtgatgatacatcatgtagacaaacttgagtcgttcctgattcatggaggtgtagttcatggtgggaagccaatttgcacacacaagtttgtagaggacttgattggacgaggtgagaaattttgatgccatgttctcccagcgattcactcgtcctccagacagaaaggtacataccttatcaatgttttcatcgcgccagtttgggtcatcctcagaaccaggaatacagtacagtgaattgataagccccggagacagcgtaaccactgaccctctgagatacaccgccacaccatcatctctctcctcagcatctggcaggttggcgacaaactcccgaatcacggttggttgaaaggcgtcggagtcaaccacggtgtagatcaaaccctaatggaacacagtttctctgacctctgcgagcttgttgttggagagaaccaaaacgctgttgtggcacaagttttttgtctcggagaatgcagtacctaccacgagcagccattgtagcaaacctacctgagaggggacgtcttgatggacggagatacgccggattagcctcatacatagcctggaacatggctcgactctcttcgtatctgggctcttttggaagaagagtctcttcgatgggaacatcagtgtggttctcaccattgctgtcatcttcagacacagaatctgcggcgacttcggttgggtcggacggagatggatgatgacgacggcgaacacgcttcttactccctctcgaggactcaccggaagttggagcattaacccaaattggttcatcgttcaccggcgagctcatagatcgactcaagcgagtgctccttcgaggcggctgcatgttggatttgtgcaagagaaccaaagtgcctcatccttaacgacaaccctaagaataagtcactcagacattgggccgttagtaacaacacaaaagcccataagacttaacaataacccaagctagaatcaggtccattagcatcaaatttttgaccaagtgttagagacattcacttacagaggcaagagaatcagagagagataaggacttcgagcgaagcacacggacaacagatacttagtaaccataggagactgtacatttggacacgacacgcagaggtgccctttgttcaagagaagaaccactgacattattgctgactcataggtattcatcaaaagaatttcaagagtaaatcaatgacctcaacccaagtctgtagctgtttctcagccgagtagctgtcacaggcgagatttccccaaaaactgtcactcaaactcgtcctcctttataacacttatctgtatggtctggaaacactgcaagtatcttgtgtgtacttcttcttctcagcacgaccataataatctcaagattcaccaagagatcacttaccattcttaatgaagaaactgatcaagtccagagagtaagtgttgtaacacccaactccggctgggttacttcaaatggtagattctttctctgcctagtccggacagtgatcgaactcagagtcagagtgtgatagcaccctgctccgacacgagtatccatcattccaggcctttattttgaagtctcatgtatccagtttcatgtgttactcattgttttggacatgtagcacacctacttttgattggcactcaccttcaccaatgtttccttgttcaatccagacagtgatcaatgctcaaacgtagaatgttgcagcatcctagtccaagatgagtgtctttcttcatcgaaccagataagtgtgatatcaggagccgttgcgagtaactatcaaggagaaacttgttgaggcaaggttttagttgtgtgatgaaatccctacagatagcagcaactatatacagcaattctactctctcatttgaacttgttcagaggtcaatgattcccaacgccttcctaagaccaagaaaggtgttgtaatccaatggttttgtgaaaagatcagccagttgtctttctgttggcacatgctcaagcgttacaatcctcatctcaaccaattctctcacaaagtgatgcctaatatccacatgtttggtgcgtgaatgctgaacaggattcttagacagatttatagcactcatgttatcacagtgaacaagcatagattccgaaatgataccataatcaactagcatctgtcgcatccagagtagttgagtgcaacaactccccaaggctattatactctgcctcggctgtcgatagagagacacaattttgcttcttgctgtgccatgacaccatgtttgtttcctaggaagaagcatccttcctgacgtgcttgtgtctatcatctaaacaccctgcccaatcggcatcgcaaaatcctgcaagattcacgttagtttcaaaagtgtaatgaaggccaaagtccaatgtacctttcacatatttgattattcgtttgacagcgttcatgtgggattccttcggatgagcctggtatcgtgcacatattcctacgcttaagcaaagatccggtctgcttgcagtgagatatagcagacttccaatcatggctctgtagagcttttcatcaactggttttccatcttcgtctctcgaaagtttagtggatgtgctcatcggggtcttagcaattttgctggtttgcattccaaatcgtttgataaggttcttggcatatgtactttgagacaccgtgataccatcagggagctgtttgacttgtaaaccgagaaagtaactcagttcacctaccatgctcatctcaaactccttcgtcattgtcttgacaaactcatccaccatcgattgatctgtacttcccgaagataatgtcatcaacgtagacttgaatgattaggatgtccttcccattctctcctatgaacagagttttatctactcctcctcgctgaaagccagccttaatgagaaactcagtcagtctctcataccaagctcgtggagcttgttttagaccataaagagctttcttaagcttgtagacatgatctggaaaatgtggatcttcaaaccctttaggttgtgaaacgtaaacctcttcttgaagtatcccatttaagaaagcacttttgacgtccatttgatagagtttgattcgaagactgcaggccatgccaaatagcagtctgatcgattcaagcctagcaactggagcgaaggtctcatcgaaatccacaccttcaatctgagcatatccttgcccgacaagtcttgacttgtttctgatcacatttccttcctcatctgtcttgtttttatgaatccatttcgttccaatgatgttcacattttcaggtcttggaaccaaagcccacacatcaagtcgttcaaactgttcaagctcttggtgcattgaatcggtccagtactcatcctcaagagcttcttgtagattcttaggttcaaagagagacacaaagcactcaactttattcaactcctccacgaagaacgccagcttcaccatttctttgaagtcaatctttttcttcctggtgactcgttcatcaaatactccaccaatgacgtcagatgaggagtgattgcgatgaacttttcctttgtctagatcgactttgatactgttctgctcattttcatctccagactcatctttgagctcggtttcagttgatgtactgacgggctgtgtcacacactcaattgtttgtgtcaccttagcttgataaaaacctatgttgtcatcaaacaccacattgacattatcaccaacaaatttggtacgctgggttgaatactctgtaagctgagctgtttgtagagtaaccgagaaacatttcaacatcactcttagcctcaaattttcctagatgatcctgatcgttcaatatgtaacagagacatccgaacacatgcatatgactcaaatttggtgtttttcctttgaagatctcatagggtgtcatcgtagtctttggctttacgtacactcgattgattatgtagcaagccgtagctacagcttctgcccagaaaccggaagggacactgtttccacataacattgctctagccatctcctgcagtgttctgttctttctttcaacaaccccgttttgttgtggagttctaggagcagcatattgatgacggattccttgactctgacagaatttatcaaactgttcattttgaaattctcctccgtgatcactcttgatctgaataatgcctccttttacttgtttgagttgcagtgccaagatacgaaagctctcaagtgcatctgatttcttcctcagaaaatcaatccaagtgtatctcgagaaatcatcaaccataacaagaatgtagcgtttaccagcaatgctctcaggtgtgattggacccataagatccatgtgtactaattcaagaacacgcttagagtgaatctctgaaatctgtttatgttgcaccttgatctgctttccctgacagcatcctccacacactgtatccgtttctttttcgagctcaggtactcctctcacaacctcagcattcactagttttgtcagtcctcgagtgttcatgtgtccaagcttcttgtgccagagatcaagtttggattctcttgcagataagcacagttgcgatggtttccacatgtagcagttgttgcctgaacgaactccatacagaactaaattccctttcgcatcaacagctctgcattctttactgttgaaaatgacttccaatccatcatcacacagttggctcacactgatcagatttgccttgagtccatccactaggtagacatttatgagacgaggaagatctggtctttccagtactccaacaccacggattcttccctgaccaccatctccaaaagtgacttttcctccttgaagaagctcaagtttctcaacaaattccagctttccagtcatatgtttggagcaaccactgtcaaagtaccacggggtatcatttgcattgctactctcagcactagtgtacgcaacattactcacaaactcgtcctcactatgtattcgtgcaaggttgcactcaatttcagtctctggattggtgtgtagctcatctcgttcgtcatgtgacacaacttgttgtacttctttatagtttgggtacaagtctcgcttggctatccagacatgaccgtagagagttggttccatgaagcacagatttaacctccacgctctctcatactgatgtcttcggaagtaacaatacttaacatgatgtccacgttttccacagaactgacatgcatttcttcgatgtggaatctgtactgagttcgtgtttttttctttctgagtggaagctctcatgatgctgacttgaggaagttttgcagatactggtttctcttcttctacagcttcttttacaaagacaacttgttttcccttctcttcagctgtatacttggtaccttgatacccgagacccttgttggtgctaggacattgtcctatagtaagaagatgatccagtgttgatgtgcctgaggtgagcattttaactttcttcagattttctgcaagttgattctgaagaagtttactctttatccatctcttctgctagtagattggtcagttgttcaatcttgacttcaaacaaatttctgacatctctcttgttctaccactgctcgtttcaaagacagaacctcttgatcagattccttcagaattgagagattggagctcttacccgatggttgttctagctccagaatgttcacttgtgctttcaacattgccttgtctttgagaagttgcaagttttcatggctaagctcagcaaatttgtcaaagagagtcttgtactctgtttcaagatcctgaccaagagtgttgtcatcttcttcatcgcctgattctgatttctcatcatcttcctgtccaattaaagccatgaaatttagatgaagttcttcttcctcactgtcattatcagactcagagtcactgaaacacacaagcgatttgtccttcttcaaagtgttaggacactcactcctagtatggccaattcccttacattcaatacacttcagctcctttctcttagtaaggggacattcattcttgaagtgaccgtatccttcacactcatgacactgaagatccttcttcttagatgttcttgatgcatcttgttttgagtattgagaactgcttcgatcagattcccctcgctgaaaacgattatttgagcgacctgttcctttctccatgcgtttcacaaacttgttgaagtttcgagccaattaatcctagattctcttcaatcttggtaactctatcactttccttagagtcagcagagaaggcaaatactcttctgagaatttgatgctcgatcagatttttccaaagtcgtgaaccttcagtatcccagataactgatcaaacttcatttcatccgtgtctactgcaatgtttagaacagctttgtatgcatcaaacctaggaggtaggcatctcagtaatttcttcactaagtctttctcctcgtacttcttaccaagaactgaagactcattagcaatctcgctaatcttggagataaaaccatcaataggatcatcatctcccattcgcagattttcgaacctagatgcaagatgatcaattcgtgtacgtcgtacactggtgtttccttcaaaatgatttatcagggtatcccaagcttccttggcagactcgcatccttgtatgatcttaaattgatccagatcaactgatgaaaagatcacagtcaaagctttagaattgaattttgatgcagctttttctgcttcagtccattgatccttaggtttaggacccagagttttatcttccatcataactgtgggagctgaccatccttcttcaacggcagtccatgcatcttcgttaatcgccctgatcacatgtctcattctggccttccagtgaccaaagtttccaccgtctaacatgattggtttgtgtaaacgtgatgagttccttcatactgtccatgatctccgcagggatcttcacctgttagtggtattagataccacagaggttccctgctctgataccaaatgaaagttcagggacaataagttaataaggaacgcacagaacttccaagaacacttccttgattaatcagaaaccttttaaacaatcttcctagatctgtttaatccgatttatactcagtcacacacgactagagacggaccagttactaatcttgtcactcgacaataacacaggacaagctatcccaacctggatactctcctattagactgcttctcaagaacactcttcttgcttaagcacgtcaaaacactaaaaaccgtaacctaaatctacccaaggtaatttggttatatacacctctcacaatatctcccctgagatatcgtgatatatacaaatccccatcgttattcatctctccgagatatcaggttcaaacgattgcctcgtcgccacgtcatcattcagctactttctcgcgtcaccatgtgttacacaacgctccatatgtgtaactcactaatcttgttccttgtctcacaagcactaatccttggtgctttcaaggatctgcccgccgttttggggtaaggtttcactagtgtctggtttttaacacggtctgtccgttgttacgtgacagtttgattaattaatcatgcatggatattatagttttattggatttacaaatgatttagatgtgttagtgaatactaaatagctattgcttagttacaatagctaggaatgaaaactaaaggtgatgctaactaagcatggaaaacatttgatgtggcatgcagggaggttatattggagtactcaaaggaaatgacgagtttaggtgaattgatctttgagcttctatcagaggctttgggattagacactaattatttcaaggatttggattgcgtcaagtctcagatgatgttgggccaatattatccaccttgccctcagcctgaccttactttaggcttaagcaagcacagtgatttttcttttctcactgttcttcttcaagacaatataggagggcttcaagttctccatgacgatgcctggtttgatgttcctcctgtccctggatgttttgtcgttaacattggagatcttctacaggttaagatcataaactgctgtggcttgtaattaacggttttaaacataaaatatatatgaagctcttttgttgttgttgtgtgcagttcataaccaatgacatgttcataagcgcggagcatagggtgctagcgcatactgcttctgtaccgagggtttcagtgccatatttcttcaccacgttcaagaaggtgaatcctcgtgtatatggacccatcaaagagctcctctcagaagataaccctcgcaagtatagagactgttccatgaccgagatctcagagatcttcagttcaaatgagatcaccattcctaggttacaccagctcaggatctgaaacatcaatcattgctataatctctccaactctatgttatgtttggtcttcttagttcctcttaaaatatctgccgcaataactacttcctcttcttctctcgaactgtgtttgaaagatatctgaaaatcttcgaataaggtgttattttgtaattgagttctttagttattaagtaagttatcatgttgtatcaaaagttgtgtgtaagagtcgtgactttagattggagaattgtgtctcttattc

SEQ ID NO: 3 - DNA, 2662 bp, Raphanus sativus var. longipinnatus MR050E
atagcattagaaaagcttttaaagaaacaatgtccagcaacgtcgttagtggggtcgaaatggatggttcgaatgaaagaaaagttttcgacgataagaaaatgggcgtaaaagccctggcggatgcaggaatcaaagagctcccagccatgttccgtgcacctccgagtattttagaaagcctgaaagcagcacgagcttctcaggatgcgaacctcttcccgaccattgatctgaaaggagtgagcctgcactacaaggatcaagatttgatgacgcggcggaacgtggtggagcagatcagagatgcgtctgcgaaatggggtttcttccgagtgaccaatcacgggttctcgaaggatttgcaggagaggatgcttgaagggcttcgtcggtttcacgcgcaagatccatcagttaagagacagtactacacacgtgatcacactcggaactttctttactacaccaacgtcgatctcttcacctctgattccgccagttggagagacacaactatttgttacacagccccggatcgtccccgacctgaggatctgcccgccgttttggggtaaggtttcactagtgtctggtttttaacacggtctgtccgttgttacgtgacagtttgattaattaatcatgcatggatattatagttttattggatttacaaatgatttagatgtgttagtgaatactaaatagctattgcttagttacaatagctaggaatgaaaactaaaggtgatgctaactaagcatggaaaacatttgatgtggcatgcagggaggttatattggagtactcaaaggaaatgacgagtttaggtgaattgatctttgagcttctatcagaggctttgggattagacactaattatttcaaggatttggattgcgtcaagtctcagatgatgttgggccaatattatccaccttgccctcagcctgaccttactttaggcttaagcaagcacagtgatttttcttttctcactgttcttcttcaagacaatataggagggcttcaagttctccatgacgatgcctggtttgatgttcctcctgtccctggatgttttgtcgttaacattggagatcttctacaggttaagatcataaactgctgtggcttgtaattaacggttttaaacataaaatatatatgaagctcttttgttgttgttgtgtgcagttcataaccaatgacatgttcataagcgcggagcatagggtgtgatgtagcgcaagctacgataacacaatgaatcctatttagtctgagaatacctaggatcaaagtcttcttgattcgttgagaactctcgagttctagccgtaacaaggaaataaaagggtttcaaacctctctttataaaatatcaatatcaataaaactgattacaagtctaagcataaaacacctatttatatgaaaaccaaataatctaaaaactattaaaatccttaagataattataactaattaagataaatatcaaaacaaataataaactagataattaagatatttggaatatatctaaatatctctctgcatcattctctccgggttggagaaagattcgtcctcgaatcttaaccgtggtcttcgattaattttcctcttttataccatatataaaataattcttcaagccatcgaatatattgatcctttgtataaaatccttttgtttgccaaaaaacaattacgccacatggcataatcaattgcctctttgcccttttgaacttcacagcaaatttagatgaagaatccaatacgtcttcaacaccttgcggtggagcaagtgccgaccaattaccttctttagaccttgatatttgacttttgacagcaccaaaaaagtctctcacgtgcttgtttgttgacttgaacacatccttgtataatatcaaatcgaccggtttgataataatcacgtcttcaccgtattgatcatatattgcaggaccaataatctctttgtactctccgtacaaatctgttgtcgaattaaatggatattcatccgtttgataaaaacacccaaacgacatggtattgaacgaagcagaatcaactttgatttagaaaaccaaagtgttggctctgataccaaactgatgtagcgcaagctacgataacacaatgaatcctatttagtctgagaatacctaggatcaaagtcttcttgattcgttgagaactctcgagttctagccgtaacaaggaaataaaagggtttcaaacctctctttataaaatatcaatatcaataaaactgattacaagtctaagcataaaacacctatttatatgaaaaccaaataatctaaaaactattaaaatccttaagataattataactaattaagataaatatcaaaacaaataataaactagataattaagatatttggaatatatctaaatatctctctgcatcagggtgctagcgcatactgcttctgtaccgagggtttcagtgccatatttcttcaccacgttcaagaaggtgaatcctcgtgtatatggacccatcaaagagctcctctcagaagataaccctcgcaagtatagagactgttccatgaccgagatctcagagatcttcagttcaaatgagatcaccattcctaggttacacca

SEQ ID NO: 4 - DNA, 20 bp, Artificial Sequence
  reverse primer
tccaggttgggatagcttgt

SEQ ID NO: 5 - DNA, 20 bp, Artificial Sequence
  reverse primer
tgaaaccttaccccaaaacg

SEQ ID NO: 6 - DNA, 21 bp, Artificial Sequence
  forward primer
gcaggagaggatgcttgaagg